(19) United States
(12) Patent Application Publication
(10) Pub. No.: US 2022/0189141 A1
(43) Pub. Date: Jun. 16, 2022

(54) IMAGE PROCESSING APPARATUS, IMAGE PROCESSING METHOD, AND STORAGE MEDIUM

(71) Applicant: CANON KABUSHIKI KAISHA, Tokyo (JP)

(72) Inventor: Naoto Takahashi, Kanagawa (JP)

(21) Appl. No.: 17/683,394

(22) Filed: Mar. 1, 2022

Related U.S. Application Data
(63) Continuation of application No. PCT/JP2020/028197, filed on Jul. 21, 2020.

(30) Foreign Application Priority Data
Sep. 6, 2019 (JP) 2019-163273

(51) Int. Cl. G06V 10/764; G06V 10/82; G06V 10/44; G06T 7/00

(52) U.S. Cl. CPC G06V 10/764; G06V 10/82; G06V 10/454; G06T 2207/30008; G06T 2207/20084; G06T 2207/20081; G06T 2207/10116; G06T 7/0012

(57) ABSTRACT

An image processing apparatus divides a radiographic image obtained through radiography into a plurality of areas, extracts, as a target area, at least one area to serve as a reference, from the plurality of areas divided, determines a rotation angle from the target area extracted, and rotates the radiographic image on the basis of the rotation angle determined.

### CROSS-REFERENCE TO RELATED APPLICATIONS

This application is a Continuation of International Patent Application No. PCT/JP2020/028197, filed Jul. 21, 2020, which claims the benefit of Japanese Patent Application No. 2019-163273, filed Sep. 6, 2019, both of which are hereby incorporated by reference herein in their entirety.

### BACKGROUND OF THE INVENTION

### Field of the Invention

The present invention relates to a technique for correcting rotational misalignment in an image obtained by radiography.

### Background Art

Digital imaging is increasingly being used in the field of medicine, and radiography devices using flat-panel detectors (called “FPDs” hereinafter) that indirectly or directly convert radiation (X-rays or the like) into electrical signals have become the mainstream. In recent years, cassette-type FPDs offering excellent portability due to their light weight and wireless implementation have arrived, enabling imaging in a more flexible arrangement.

Incidentally, in imaging using a cassette-type FPD, the subject can be positioned freely with respect to the FPD, and thus the orientation of the subject in the captured image is indeterminate. It is therefore necessary to rotate the image after capture such that the image has the proper orientation (e.g., the subject's head is at the top of the image). In addition to cassette-type FPDs, stationary FPDs can also be used for imaging in positions such as upright, reclining, and the like, but since the orientation of the subject may not be appropriate depending on the positioning of the FPD, it is necessary to rotate the image after the image is captured.

Such an image rotation operation is extremely complicated and leads to an increased burden on the operator. Accordingly, a method of automatically rotating images has been proposed. For example, PTL 1 discloses a method in which rotation and flipping directions are determined using user-input information such as the patient orientation, the visual field position of radiography, and the like, and then performing processing for at least one of rotating and flipping the image in the determined direction. PTL 2, meanwhile, discloses a method of extracting a vertebral body region from a chest image and rotating the chest image such that the vertebral body direction is vertical. Furthermore, PTL 3 discloses a method for obtaining the orientation of an image by classifying rotation angles into classes.

### CITATION LIST

### Patent Literature

PTL 1: Japanese Patent Laid-Open No. 2017-51487
PTL 2: Japanese Patent No. 5027011
PTL 3: Japanese Patent Laid-Open No. 2008-520344

However, although the method of PTL 1 can rotate images according to a uniform standard using user-input information, there is a problem in that the method cannot correct for subtle rotational misalignment that occurs with each instance of imaging due to the positioning of the FPD. In addition, the method of PTL 2 is based on the properties of chest images, and there is thus a problem in that the method cannot be applied to various imaging sites other than the chest. Furthermore, although the method of PTL 3 obtains the orientation of the image from a region of interest, the method of calculating the region of interest is set in advance. There is thus a problem in that the method cannot flexibly handle user preferences and usage environments. The criteria for adjusting the orientation of the image varies depending on the user, such that, for example, when imaging the knee joint, the image orientation may be adjusted on the basis of the femur, on the basis of the lower leg bone, or the like. As such, if the region of interest differs from the area that the user wishes to use as a reference for image orientation adjustment, the desired rotation may not be possible.

### SUMMARY OF THE INVENTION

In view of the foregoing problems, the present disclosure provides a technique for image rotational misalignment correction that can handle a variety of changes in conditions.

According to one aspect of the present invention, there is provided an image processing apparatus comprising: a dividing unit configured to divide a radiographic image obtained through radiography into a plurality of areas; an extracting unit configured to extract, as a target area, at least one area to serve as a reference, from the plurality of areas divided; a determining unit configured to determine a rotation angle from the target area extracted; and a rotating unit configured to rotate the radiographic image on the basis of the rotation angle determined.

According to another aspect of the present invention, there is provided an image processing method comprising: determining information about a rotation angle using a target area in a radiographic image obtained through radiography; and rotating the radiographic image using the determined information.

According to another aspect of the present invention, there is provided an image processing apparatus comprising: a determining unit configured to determine information about a rotation angle using a target area in a radiographic image obtained through radiography; and a rotating unit configured to rotate the radiographic image using the determined information.

Further features of the present invention will become apparent from the following description of exemplary embodiments with reference to the attached drawings.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a diagram illustrating an example of the overall configuration of a radiography device according to a first embodiment.

FIG. 2 is a flowchart illustrating a processing sequence of image processing according to the first embodiment.

FIG. 3 is a diagram illustrating an example of the overall configuration of a radiography device according to a second embodiment.

FIG. 4 is a flowchart illustrating a processing sequence of image processing according to the second embodiment.

FIG. 5A illustrates an example of a relationship between classes and labels.

FIG. 5B illustrates an example of information associated with an imaging protocol.

FIG. 6 is a diagram illustrating an example of target area extraction processing.

FIG. 7 is a diagram illustrating an example of major axis angle calculation processing.

FIG. 8 is a diagram illustrating the orientation of a major axis.

FIG. 9 is a diagram illustrating an example of operations in setting a rotation direction.

FIG. 10 is a diagram illustrating an example of operations in setting a rotation direction.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

### First Embodiment

Configuration of Radiography Device

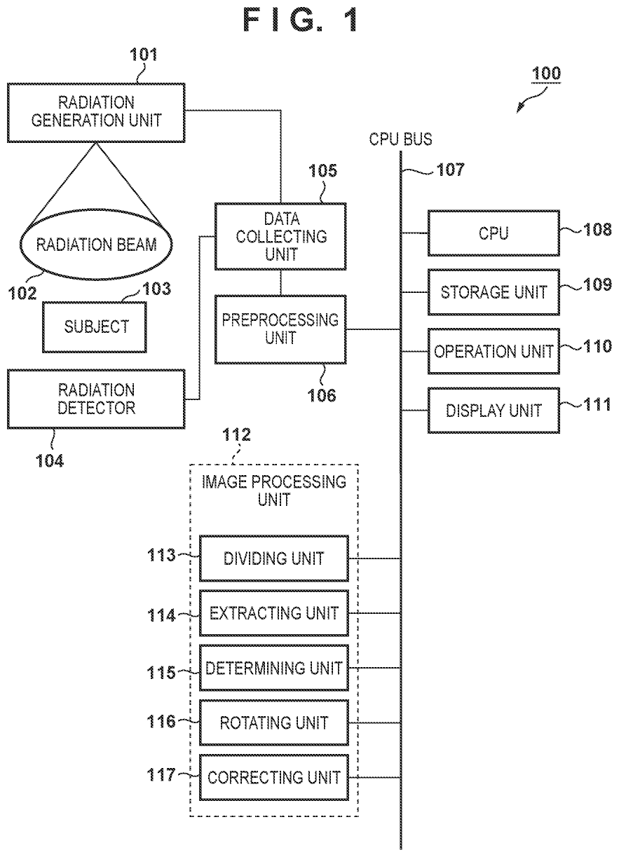

FIG. 1 illustrates an example of the overall configuration of a radiography device 100 according to the present embodiment. The radiography device 100 includes a radiation generation unit 101, a radiation detector 104, a data collecting unit 105, a preprocessing unit 106, a Central Processing Unit (CPU) 108, a storage unit 109, an operation unit 110, a display unit 111, and an image processing unit 112, and these constituent elements are connected to each other by a CPU bus 107 so as to be capable of exchanging data with each other. The image processing unit 112 has a role of correcting rotational misalignment in a radiographic image obtained through radiography, and includes a dividing unit 113, an extracting unit 114, a determining unit 115, a rotating unit 116, and a correcting unit 117.

The storage unit 109 stores various types of data necessary for processing performed by the CPU 108, and functions as a working memory of the CPU 108. The CPU 108 controls the operations of the radiography device 100 as a whole and the like. An operator makes imaging instructions to the radiography device 100 by using the operation unit 110 to select one desired imaging protocol from among a plurality of imaging protocols. The processing of selecting the imaging protocol is performed, for example, by displaying a plurality of imaging protocols, which are stored in the storage unit 109, in the display unit 111, and having the operator (user) select a desired one of the displayed plurality of imaging protocols using the operation unit 110. When an imaging instruction is made, the CPU 108 causes radiography to be performed by controlling the radiation generation unit 101 and the radiation detector 104. Note that the selection of the imaging protocol and the imaging instruction to the radiography device 100 may be made through separate operations/instructions by the operator.

The imaging protocols according to the present embodiment will be described here. “Imaging protocol” refers to a set of a series of operating parameters used when performing a desired examination. By creating a plurality of imaging protocols in advance and storing the protocols in the storage unit 109, the operator can easily select conditions for settings according to the examination. In information of the imaging protocol, various types of setting information, such as image processing parameters and the like, are associated with imaging sites, imaging conditions (tube voltage, tube current, irradiation time, and the like), and the like, for example. Note that in the present embodiment, information pertaining to the rotation of an image is also associated with each imaging protocol, and the image processing unit 112 corrects rotational misalignment of the image by using the information pertaining to the rotation of that image. The rotational misalignment correction will be described in detail later.

In the radiography, first, the radiation generation unit 101 irradiates a subject 103 with a radiation beam 102. The radiation beam 102 emitted from the radiation generation unit 101 passes through the subject 103 while being attenuated and reaches the radiation detector 104. The radiation detector 104 then outputs a signal according to the intensity of the radiation that has reached the radiation detector 104. Note that in the present embodiment, the subject 103 is assumed to be a human body. The signal output from the radiation detector 104 is therefore data obtained by imaging the human body.

The data collecting unit 105 converts the signal output from the radiation detector 104 into a predetermined digital signal and supplies the result as image data to the preprocessing unit 106. The preprocessing unit 106 performs preprocessing such as offset correction, gain correction, and the like on the image data supplied from the data collecting unit 105. The image data (radiographic image) preprocessed by the preprocessing unit 106 is sequentially transferred to the storage unit 109 and the image processing unit 112 over the CPU bus 107, under the control of the CPU 108.

The image processing unit 112 executes image processing for correcting rotational misalignment of the image. The image processed by the image processing unit 112 is displayed in the display unit 111. The image displayed in the display unit 111 is confirmed by the operator, and after this confirmation, the image is output to a printer or the like (not shown), which ends the series of imaging operations.

Flow of Processing

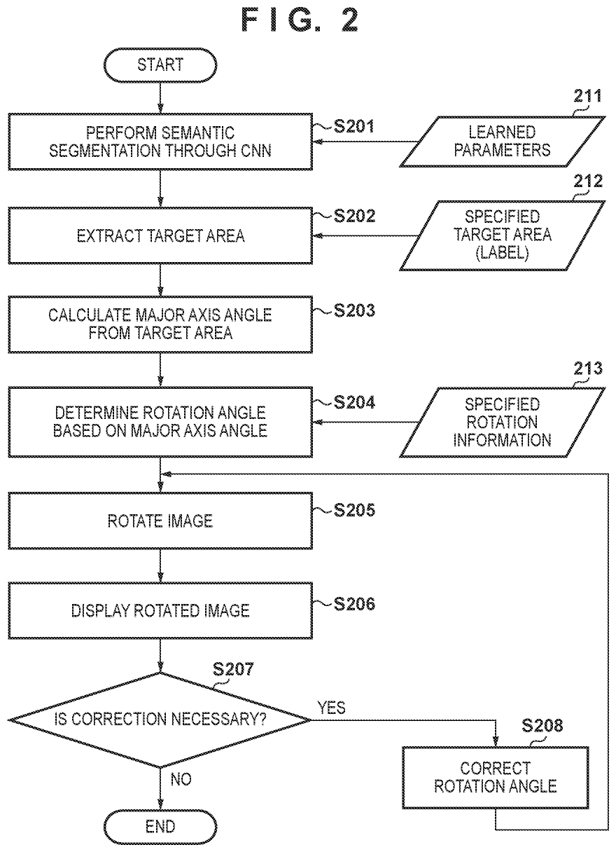

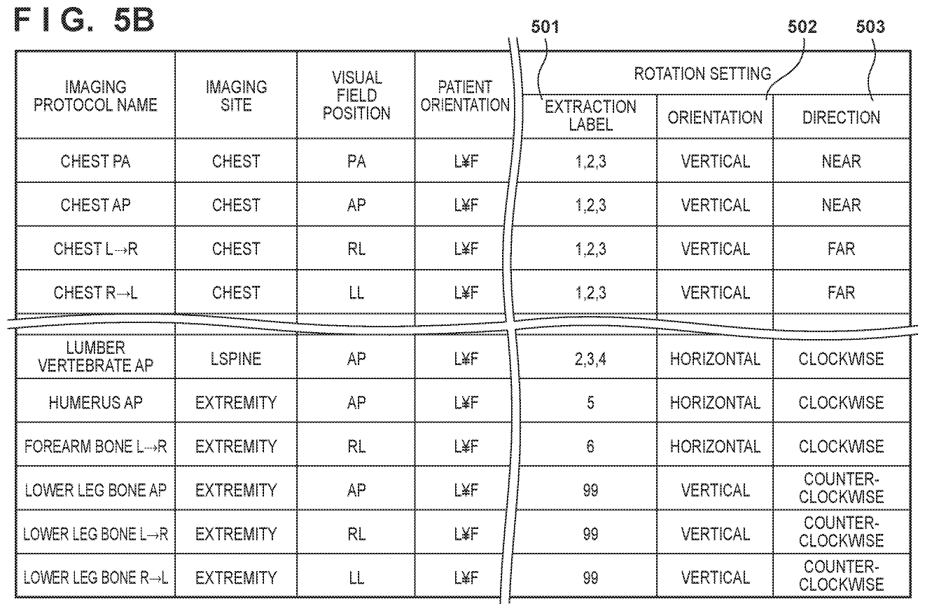

The flow of processing by the image processing unit 112 in the radiography device 100 will be described next with reference to FIG. 2. FIG. 2 is a flowchart illustrating a processing sequence performed by the image processing unit 112 according to the present embodiment. The flowchart in FIG. 2 can be realized by the CPU 108 executing a control program stored in the storage unit 109, and computing and processing information as well as controlling each instance of hardware. The processing in the flowchart illustrated in FIG. 2 starts after the operator selects an imaging protocol and makes an imaging instruction through the operation unit 110, and the image data obtained by the preprocessing unit 106 is transferred to the image processing unit 112 via the CPU bus 107 as described above. Note that the information illustrated in FIGS. 5A and 5B (where FIG. 5A is an example of a relationship between classes and labels, and FIG. 5B is an example of information associated with an imaging protocol) is assumed to be stored in the storage unit 109 in advance.

In S201, the dividing unit 113 divides an input image (also called simply an “image” hereinafter) into desired areas and generates a segmentation map (a multivalue image). Specifically, the dividing unit 113 adds, to each pixel in the input image, a label indicating a class to which the pixel belongs (e.g., an area corresponding to an anatomical classification). FIG. 5A illustrates an example of a relationship between the classes and the labels. When using the relationship illustrated in FIG. 5A, the dividing unit 113 gives a pixel value of 0 to pixels in an area belonging to the skull, and a pixel value of 1 to pixels in an area belonging to the cervical spine, in the captured image. The dividing unit 113 provides labels corresponding to the areas to which pixels belong for other areas as well, and generates the segmentation map.

Note that the relationship between the classes and the labels illustrated in FIG. 5A is just an example, and the criteria, granularity, or the like with which the image is divided are not particularly limited. In other words, the relationship between the classes and labels can be determined as appropriate according to an area level serving as a reference when correcting rotational misalignment. Areas other than the subject structure may also be labeled in the same way, e.g., areas where radiation reaches the sensor directly, areas where radiation is blocked by a collimator, and the like can also be labeled separately, and the segmentation map can be generated.

Here, as described above, the dividing unit 113 performs what is known as “semantic segmentation” (semantic area division), in which the image is divided into desired areas, and can use a machine learning method that is already publicly-known. Note that semantic segmentation using a convolutional neural network (CNN) as the algorithm for the machine learning is used in the present embodiment. A CNN is a neural network constituted by convolutional layers, pooling layers, fully-connected layers, and the like, and is realized by combining each layer appropriately according to the problem to be solved. A CNN requires prior training. Specifically, it is necessary to use what is known as “supervised learning” using a large amount of training data to adjust (optimize) parameters (variables) such as filter coefficients used in the convolutional layers, weights and bias values of each layer, and the like. In supervised learning, a large number of samples of combinations of input images to be input to the CNN and expected output results (correct answers) when given the input images (training data) are prepared, and the parameters are adjusted repeatedly so that the expected results are output. The error back propagation method (back propagation) is generally used for this adjustment, and each parameter is adjusted repeatedly in the direction in which the difference between the correct answer and the actual output result (error defined by a loss function) decreases.

Note that in the present embodiment, the input image is the image data obtained by the preprocessing unit 106, and the expected output result is a segmentation map of correct answers. The segmentation map of correct answers is manually created according to the desired granularity of the divided areas, and training is performed using the created map to determine the parameters of the CNN (learned parameters 211). Here, the learned parameters 211 are stored in the storage unit 109 in advance, and the dividing unit 113 calls the learned parameters 211 from the storage unit 109 when executing the processing of S201 and performs semantic segmentation through the CNN (S201).

Here, the training may be performed by generating only a single set of learned parameters using data from a combination of all sites, or may be performed individually by dividing the training data by site (e.g., the head, the chest, the abdomen, the limbs, and the like) and generating a plurality of sets of learned parameters. In this case, the plurality of sets of learned parameters may be stored in the storage unit 109 in advance in association with imaging protocols, and the dividing unit 113 may then call the corresponding learned parameters from the storage unit 109 in accordance with the imaging protocol of the input image and perform the semantic segmentation using the CNN.

Note that the network structure of the CNN is not particularly limited, and any generally-known structure may be used. Specifically, a Fully Convolutional Network (FCN), SegNet, U-net, or the like may be used. Additionally, although the present embodiment describes the image data obtained by the preprocessing unit 106 as the input image input to the image processing unit 112, a reduced image may be used as the input image.

Next, in S202, on the basis of the imaging protocol selected by the operator, the extracting unit 114 extracts an area to be used to calculate (determine) the rotation angle (an area serving as a reference for rotation) as a target area. FIG. 5B illustrates an example of information associated with the imaging protocol, used in the processing in S202. As the specific processing performed in S202, the extracting unit 114 calls information 212 of the target area (an extraction label 501) specified by the imaging protocol selected by the operator, and generates, through the following formula, a mask image Mask having a value of 1 for pixels corresponding to the number of the extraction label 501 that has been called.

$$Mask ( i , j ) = \{ \begin{matrix}1 , & Map ( i , j ) = L \\ 0 , & Map ( i , j ) \neq L\end{matrix} \qquad [ Math . 1 ]$$

Here, “Map” represents the segmentation map generated by the dividing unit 113, and “(i,j)” represents coordinates (ith row, jth column) in the image. L represents the number of the extraction label 501 that has been called. Note that if a plurality of numbers for the extraction label 501 are set (e.g., the imaging protocol name “chest PA” in FIG. 5B or the like), the value of Mask is set to 1 if the value of Map corresponding to any one of the label numbers.

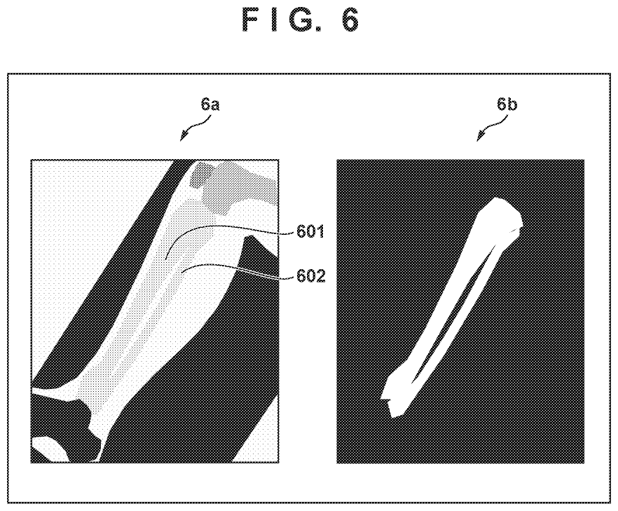

FIG. 6 illustrates an example of the target area extraction processing performed by the extracting unit 114. An image 6a represents an image captured using an imaging protocol “lower leg bones L→R” indicated in FIG. 5B. Here, the number of the extraction label 501 corresponding to “lower leg bones L→R” is 99, and this label number indicates a lower leg bone class (FIG. 5A). Accordingly, in the segmentation map of this image, the values of the tibia (an area 601 in the image 6a) and the fibula (an area 602 in the image 6a), which are the lower leg bones, are 99. Accordingly, a mask image in which the lower leg bones are extracted can, as indicated by an image 6b, be generated by setting the values of pixels for which the value is 99 to 1 (white, in the drawing) and setting the values of other pixels to 0 (black, in the drawing).

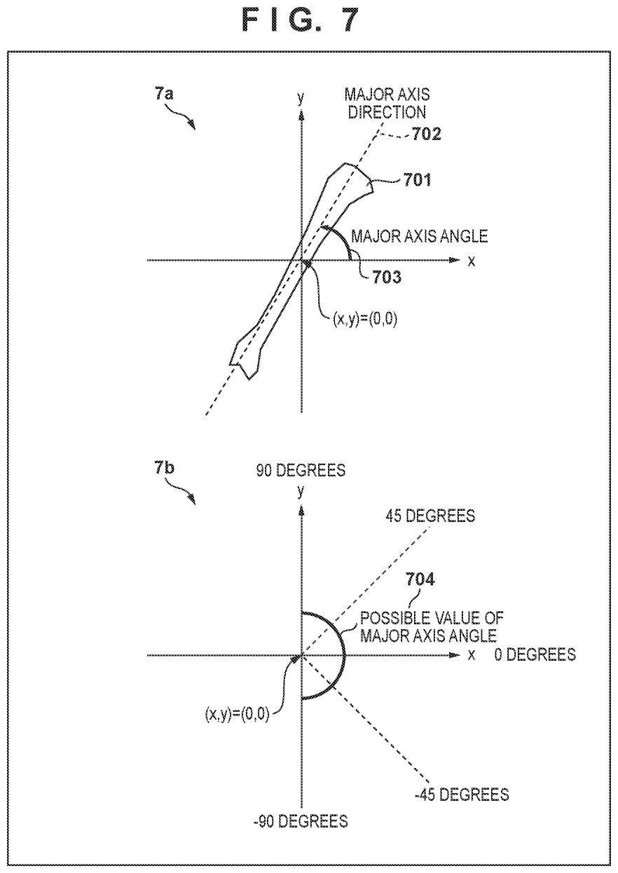

Next, in S203, the determining unit 115 calculates a major axis angle from the extracted target area (i.e., an area in which the value of Mask is 1). FIG. 7 illustrates an example of the major axis angle calculation processing. In coordinates 7a, assuming the target area extracted in S202 is an object 701, the major axis angle corresponds to an angle 703 between the direction in which the object 701 is extending, i.e., a major axis direction 702, and the x axis (the horizontal direction with respect to the image). Note that the major axis direction can be determined through any well-known method. Additionally, the position of an origin (x,y)=(0,0) may be specified by the CPU 108 as a center point of the object 701 in the major axis direction 702, or may be specified by the operator making an operation unit the operation unit 110. The position of the origin may be specified through another method as well.

The determining unit 115 can calculate the angle 703 (i.e., the major axis angle) from a moment feature of the object 701. Specifically, a major axis angle A [degrees] is calculated through the following formula.

$$\{ \begin{matrix}\frac{180}{π} \cdot {tan}^{- 1} ( \frac{{M}_{0 , 2} - {M}_{2 , 0} + \sqrt{{( {M}_{0 , 2} - {M}_{2 , 0} )}^{2} + 4 \cdot {M}_{1 , 1}^{2}}}{2 \cdot {M}_{1 , 1}} ) , & {M}_{0 , 2} > {M}_{2 , 0} \\ \frac{180}{π} \cdot {tan}^{- 1} ( \frac{2 \cdot {M}_{1 , 1}}{{M}_{2 , 0} - {M}_{0 , 2} + \sqrt{{( {M}_{2 , 0} - {M}_{0 , 2} )}^{2} + 4 \cdot {M}_{1 , 1}^{2}}} ) , & otherwise\end{matrix} \qquad [ Math . 2 ]$$

Here, M_{p,q} represents a p+q-order moment feature, and is calculated through the following formula.

$${M}_{p , q} = {\sum }_{i = 0}^{h - 1} {\sum }_{j = 0}^{w - 1} {x}_{j}^{p} \cdot {y}_{i}^{q} \cdot Mask ( i , j ) \\ {x}_{j} = j - ( {\sum }_{i = 0}^{h - 1} {\sum }_{k = 0}^{w - 1} k \cdot Mask ( i , k ) ) / ( {\sum }_{i = 0}^{h - 1} {\sum }_{k = 0}^{w - 1} Mask ( i , k ) ) \\ {y}_{i} = - i + ( {\sum }_{k = 0}^{h - 1} {\sum }_{j = 0}^{w - 1} k \cdot Mask ( k , j ) ) / ( {\sum }_{k = 0}^{h - 1} {\sum }_{j = 0}^{w - 1} Mask ( k , j ) ) \qquad [ Math . 3 ]$$

Here, h represents a height [pixels] of the mask image Mask, and w represents a width [pixels] of the mask image Mask. The major axis angle calculated as indicated above can take on a range of from −90 to 90 degrees, as indicated by an angle 704 in coordinates 7b.

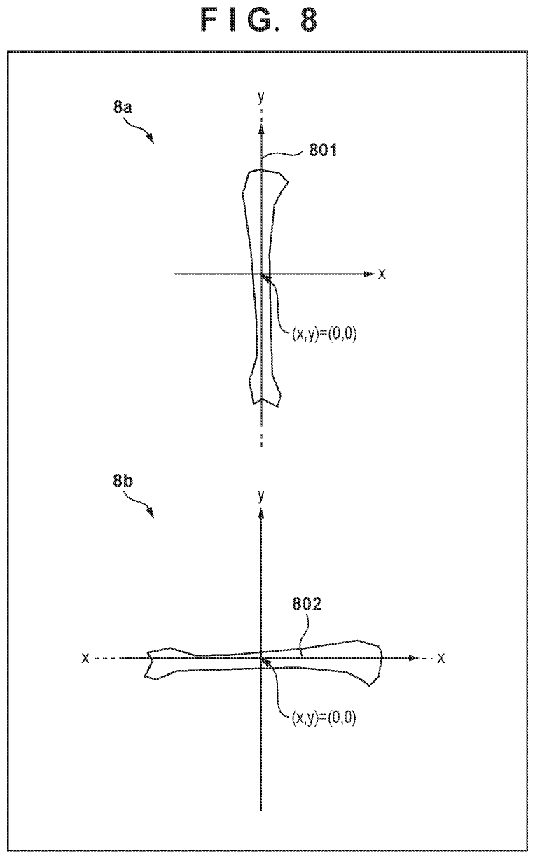

Next, in S204, the determining unit 115 determines the rotation angle of the image on the basis of the major axis angle. Specifically, the determining unit 115 calls rotation information (setting values of an orientation 502 and a rotation direction 503 of the major axis in FIG. 5B) 213, specified by the imaging protocol selected by the operator, and calculates the rotation angle using that information. The orientation of the major axis is indicated in FIG. 8. When the orientation 502 of the major axis is set to “vertical” (i.e., the vertical direction with respect to the image), the determining unit 115 calculates a rotation angle for setting the major axis to the up-down direction (coordinates 8a). On the other hand, when the orientation of the major axis is set to “horizontal” (i.e., the horizontal direction with respect to the image), the determining unit 115 calculates a rotation angle for setting the major axis to the left-right direction (coordinates 8b).

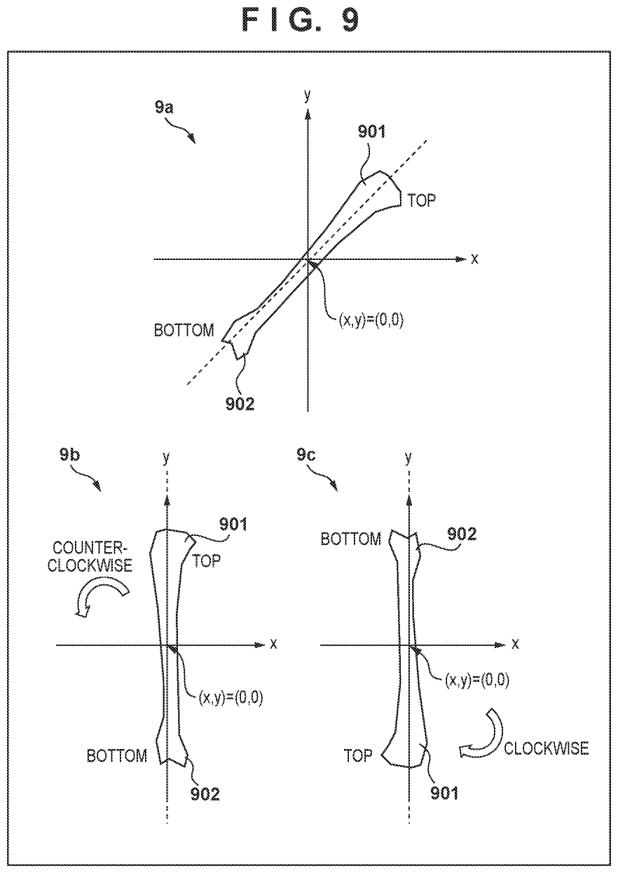

Note that the rotation direction 503 sets whether the image is to be rotated counterclockwise or clockwise. FIG. 9 illustrates an example of operations in setting the rotation direction. For example, when the orientation 502 of the major axis is set to “vertical” and the rotation direction 503 is set to counterclockwise with respect to coordinates 9a, the determining unit 115 obtains a rotation angle that sets the major axis to “vertical” in the counterclockwise direction, as indicated in coordinates 9b. Additionally, when the orientation 502 of the major axis is set to “vertical” and the rotation direction 503 is set to clockwise with respect to coordinates 9a, the determining unit 115 obtains a rotation angle that sets the major axis to “vertical” in the clockwise direction, as indicated in coordinates 9c. Accordingly, in both settings, an upper part 901 and a lower part 902 of the object are rotated so as to be reversed.

The specific calculation of the rotation angle for the determining unit 115 to execute the above-described operations are as indicated by the following formula.

$$tA = \{ \begin{matrix}90 - A , & vertical and counterclockwise \\ - 90 - A , & vertical and clockwise \\ 180 - A , & horizontal and countercloskwise \\ 0 - A , & horizontal and clockwise\end{matrix} \qquad [ Math 4 ]$$

Here, A represents the major axis angle.

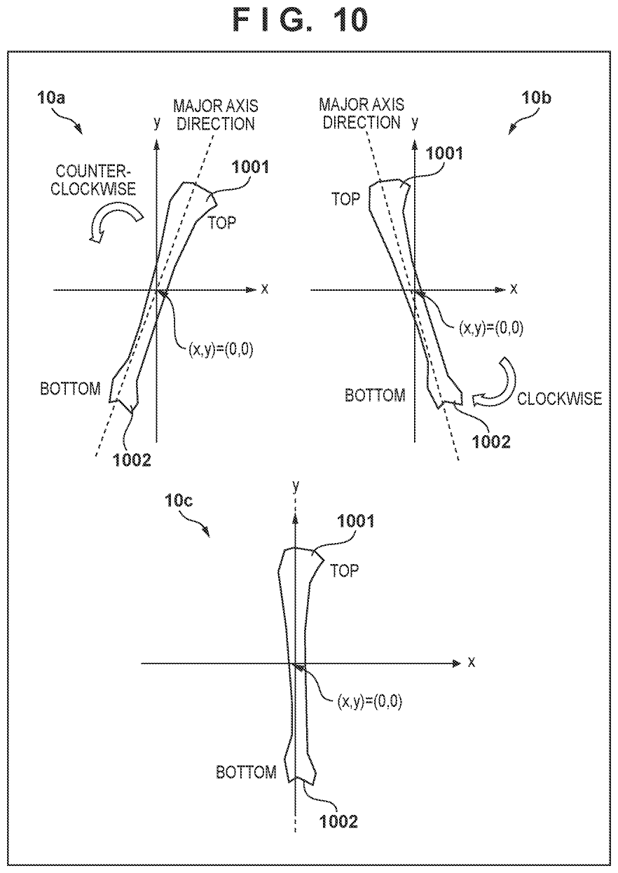

Note that in the present embodiment, “near” or “far” can also be set as the rotation direction 503. When the rotation direction 503 is set to “near”, the one of counterclockwise and clockwise which has a smaller absolute value for a rotation angle rotA obtained through the foregoing may be used as the rotation angle. Additionally, when the rotation direction 503 is set to “far”, the one of counterclockwise and clockwise which has a greater absolute value for a rotation angle rotA obtained through the foregoing may be used as the rotation angle. FIG. 10 illustrates an example of operations in setting the rotation direction. When the orientation 502 of the major axis is set to “vertical” and the rotation direction 503 is set to “near”, as indicated in coordinates 10a and coordinates 10b, the major axis is shifted slightly to the left or right relative to they axis, but the object is rotated such that an upper part 1001 thereof is at the top in both cases (coordinates 10c). This setting is therefore useful for use cases where the axis is shifted slightly to the left or right due to the positioning of the imaging (the radiation detector 104).

The method for calculating the rotation angle has been described thus far. Although the present embodiment describes calculating the rotation angle on the basis of the orientation and the rotation direction of the major axis, it should be noted that the calculation is not limited thereto. Additionally, although the orientation of the major axis is described has having two patterns, namely “vertical” and “horizontal”, the configuration may be such that any desired angles are set.

Next, in S205, the rotating unit 116 rotates the image according to the rotation angle determined in S204. Specifically, the relationship between the image coordinates (ith row, jth column) before the rotation and the image coordinates (kth row, lth column) after the rotation is indicated by the following formula.

$$[ \begin{matrix}l \\ k\end{matrix} ] = [ \begin{matrix}cos θ & sin θ \\ - sin θ & cos θ\end{matrix} ] [ \begin{matrix}j - \frac{{w}_{in} - 1}{2} \\ i - \frac{{h}_{in} - 1}{2}\end{matrix} ] + [ \begin{matrix}\frac{{w}_{out} - 1}{2} \\ \frac{{h}_{out} - 1}{2}\end{matrix} ] \\ θ = rotA \cdot \frac{π}{180} \qquad [ Math 5 ]$$

Here, wᵢₙ and hᵢₙ are a width [pixels] and a height [pixels] of the image before rotation, respectively. Additionally, wₒᵤₜ and hₒᵤₜ are a width [pixels] and a height [pixels] of the image after rotation, respectively.

The above relationship may be used to transform an image I (i,j) before rotation to an image R (k,j) after rotation. Note that in the above transformation, if the transformed coordinates are not integers, the values of the coordinates may be obtained through interpolation. Although the interpolation method is not particularly limited, a publicly-known technique such as nearest-neighbor interpolation, bilinear interpolation, bicubic interpolation, or the like may be used, for example.

Next, in S206, the CPU 108 displays the rotated image in the display unit 111. In S207, the operator confirms the rotated image, and if it is determined that no correction is necessary (NO in S207), the operator finalizes the image through the operation unit 110, and ends the processing. However, if the operator determines that correction is necessary (YES in S207), the operator corrects the rotation angle through the operation unit 110 in S208. Although the correction method is not particularly limited, for example, the operator can input a numerical value for the rotation angle directly through the operation unit 110. If the operation unit 110 is constituted by a slider button, the rotation angle may be changed in ±1 degree increments based on the image displayed in the display unit 111. If the operation unit 110 is constituted by a mouse, the operator may correct the rotation angle using the mouse.

The processing of S205 and S206 is then executed using the corrected rotation angle, and in S207, the operator once again confirms the image rotated by the corrected rotation angle to determine whether it is necessary to correct the rotation angle again. If the operator determines that correction is necessary, the processing of S205 to S208 is repeatedly executed, and once it is determined that no corrections are necessary, the operator finalizes the image through the operation unit 110, and ends the processing. Although the present embodiment describes a configuration in which the rotation angle is corrected, the image rotated the first time may be adjusted (fine-tuned) through the operation unit 110 to take on the orientation desired by the operator.

As described above, according to the present embodiment, an area serving as a reference for rotation (a target area) can be changed freely from among areas obtained through division, through association with imaging protocol information, and rotational misalignment can therefore be corrected according to a standard intended by an operator (a user).

### Second Embodiment

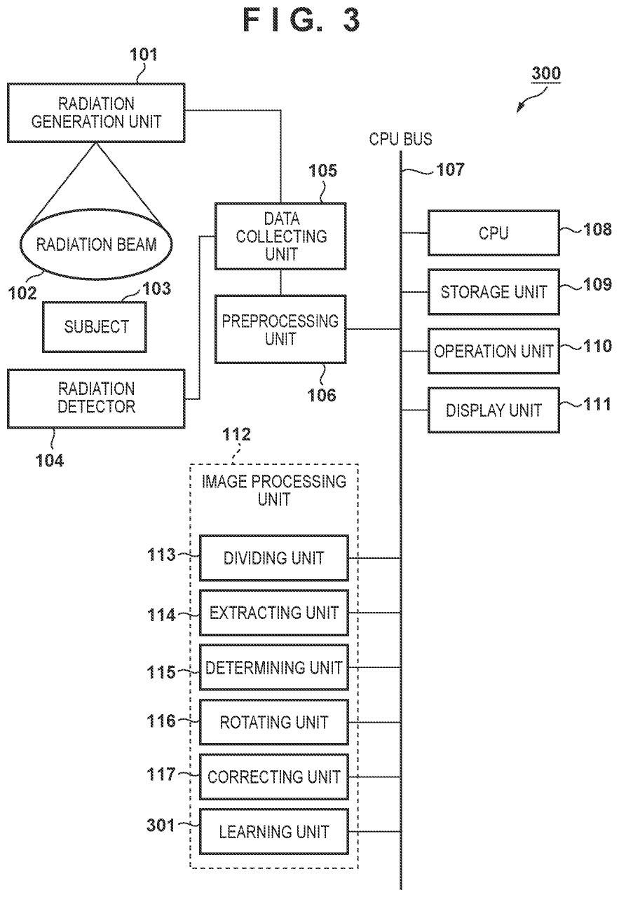

A second embodiment will be described next. FIG. 3 illustrates an example of the overall configuration of a radiography device 300 according to the present embodiment. Aside from including a learning unit 301, the configuration of the radiography device 300 is the same as the configuration of the radiography device 100 described in the first embodiment and illustrated in FIG. 1. By including the learning unit 301, the radiography device 300 can change the method for dividing the areas, in addition to the operations described in the first embodiment. The following will describe points different from the first embodiment.

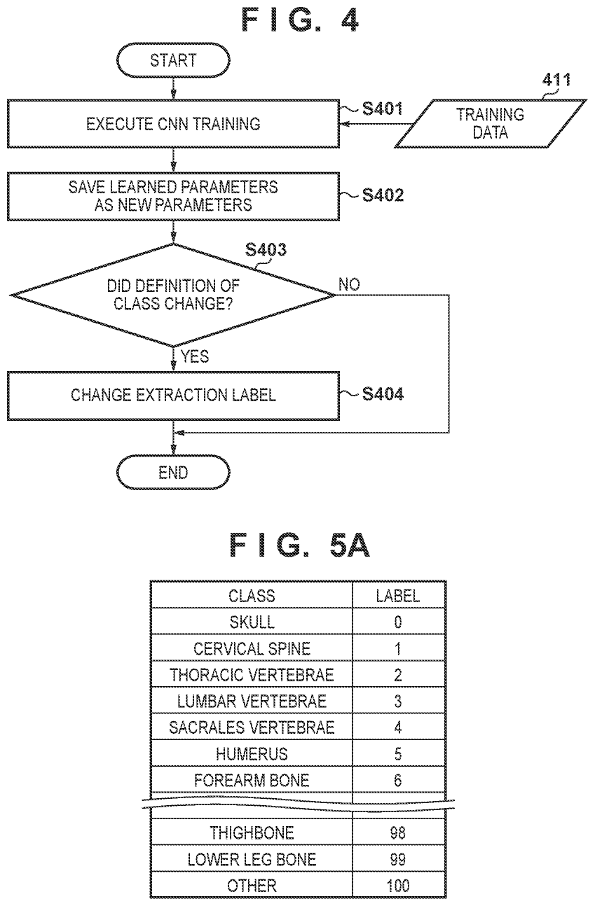

FIG. 4 is a flowchart illustrating a processing sequence performed by the image processing unit 112 according to the present embodiment. The flowchart in FIG. 4 can be realized by the CPU 108 executing a control program stored in the storage unit 109, and computing and processing information as well as controlling each instance of hardware.

In S401, the learning unit 301 executes CNN retraining. Here, the learning unit 301 performs the retraining using training data 411 generated in advance. For the specific training method, the same error back propagation (back propagation) as that described in the first embodiment is used, with each parameter being repeatedly adjusted in the direction that reduces the difference between the correct answer and the actual output result (error defined by a loss function).

In the present embodiment, the method of dividing the areas can be changed by changing the training data, i.e., the correct answer segmentation map. For example, although the lower leg bones are taken as a single area and given the same label in FIG. 5A, if the area is to be broken down into the tibia and the fibula, a new correct answer segmentation map (training data) providing different labels as separate regions may be generated in advance and used in the processing of S401. Additionally, although the cervical, thoracic, lumbar, and sacral vertebrae are taken as individual areas and given different labels in FIG. 5A, if the vertebral body is to be taken as a single region and given the same label, a new correct answer segmentation map (training data) providing different labels as separate regions may be generated in advance and used in the processing of S401.

Next, in S402, the learning unit 301 saves the parameters found through the retraining in the storage unit 109 as new parameters of the CNN (updates the existing parameters). If the definitions of the classes and the labels are changed by the new correct answer segmentation map (YES in S403), the CPU 108 changes the extraction label 501 (FIG. 5B) in S404 according to the change in the classes and the labels. Specifically, if, for example, the label assigned to the thoracic vertebrae in FIG. 5A is changed from 2 to 5, the CPU 108 changes the value of the extraction label 501 in FIG. 5B from 2 to 5.

The method of dividing the areas can be changed as described above. Note that if the parameters 211 and the label information 212 indicated in the flowchart in FIG. 2 are changed as described above for the next and subsequent instance of image capturing, the rotational misalignment can be corrected in the newly-defined area.

As described above, according to the present embodiment, the method of dividing the areas can be changed, and the operator (user) can freely change the definition of the area serving as the reference for rotational misalignment.

According to the present disclosure, a technique for image rotational misalignment correction that can handle a variety of changes in conditions is provided.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An image processing apparatus comprising:
a dividing unit configured to divide a radiographic image obtained through radiography into a plurality of areas;
an extracting unit configured to extract, as a target area, at least one area to serve as a reference, from the plurality of areas divided;
a determining unit configured to determine a rotation angle from the target area extracted; and
a rotating unit configured to rotate the radiographic image on the basis of the rotation angle determined.

2. The image processing apparatus according to claim 1, wherein each of the plurality of areas is an area corresponding to an anatomical classification.

3. The image processing apparatus according to claim 1, wherein the dividing unit divides the radiographic image into the plurality of areas using a parameter learned in advance through machine learning using training data.

4. The image processing apparatus according to claim 3, wherein an algorithm for the machine learning is a convolutional neural network (CNN).

5. The image processing apparatus according to claim 3, wherein the dividing unit divides the radiographic image into the plurality of areas using a parameter learned using training data corresponding to each of parts of the radiographic image.

6. The image processing apparatus according to claim 3, further comprising:
a learning unit configured to generate the parameter by learning using new training data obtained by changing the training data,
wherein the dividing unit divides the radiographic image into the plurality of areas using the parameter generated by the learning unit.

7. The image processing apparatus according to claim 1, wherein the extracting unit extracts the target area according to a setting made by an operator.

8. The image processing apparatus according to claim 1, wherein the determining unit determines the rotation angle on the basis of a direction of a major axis, the direction being a direction in which the target area extends.

9. The image processing apparatus according to claim 7, wherein the determining unit determines the rotation angle on the basis of a direction of a major axis of the target area and a direction of rotation set by the operator.

10. The image processing apparatus according to claim 8, wherein the determining unit determines the rotation angle such that the direction of the major axis of the target area is horizontal or vertical relative to the radiographic image.

11. The image processing apparatus according to claim 1, further comprising:
a correcting unit configured to correct the rotation angle determined by the determining unit and determining a corrected rotation angle,
wherein the rotating unit rotates the radiographic image on the basis of the corrected rotation angle.

12. An image processing method comprising:
determining information about a rotation angle using a target area in a radiographic image obtained through radiography; and
rotating the radiographic image using the determined information.

13. A non transitory computer-readable storage medium storing a program for causing a computer to execute the method according to claim 12.

14. An image processing apparatus comprising:
a determining unit configured to determine information about a rotation angle using a target area in a radiographic image obtained through radiography; and
a rotating unit configured to rotate the radiographic image using the determined information.
